(19) United States
(12) Patent Application Publication
(10) Pub. No.: US 2011/0177549 A1
(43) Pub. Date: Jul. 21, 2011

(54) METHOD, KIT AND SYSTEM FOR CULTURABLE CELL COUNT

(75) Inventors: Vladimir Glukhman, Jerusalem (IL); Elon Kaplan, Ramat Hasharon (IL)

(73) Assignee: Tacount Exact Ltd.

(21) Appl. No.: 13/003,128

(22) PCT Filed: Jul. 9, 2009
(86) PCT No.: PCT/IL2009/000690

§ 371 Date: Mar. 7, 2011

Related U.S. Application Data
(60) Provisional application No. 61/079,445, filed on Jul. 10, 2008.

(51) Int. Cl. C12Q 1/06; C12M 1/34

(52) U.S. Cl. 435/39; 435/287.1; 435/288.7

(57) ABSTRACT

The present invention provides a method, kit and system for determining a quantitative value indicative of the number of culturable microbial cells, such as E. Coli, Ps. Aeruginosa or a mixture of microbial cells (e.g. for total microbial count) in a tested sample, the method comprising (i) contacting the tested sample, susceptible of carrying culturable microbial cells with at least one signal-emitting agent capable of associating with the microbial cell's membrane, said contacting is for a predetermined first time period (T1) being sufficient for internalization of the signal-emitting agent into the microbial cell to a level at which the signal emitted from the sample essentially reaches a plateau; (ii) removing from said tested sample non-internalized signal emitting agent; (iii) during a second time period (T2), following said first time period (T1), and at which said signal is essentially maintained in said plateau, detecting from signal emitting objects within said sample signal emitting culturable cells, said detection being based on selection parameters predetermined for said culturable cells; and (iv) determining, based on said selected signal emitting objects, a quantitative value indicative of the number of culturable cells in the tested sample. The kit comprises one or more of said signal emitting agents and instructions for use of same. Also provided is a program storage device readable by machine, tangibly embodying a program of instructions executable by the machine to perform said method and a computer program comprising computer program code means for performing said method.

### FIELD OF THE INVENTION

The present invention relates to methods, kits and systems for quantitative measuring an amount of cells in a sample.

### PRIOR ART

The following is a list of art which is considered to be pertinent for describing the state of the art in the field of the invention.
(1) International Patent Application Publication No. WO06/065350 (Kimberly Clark Worldwide Inc.)
(2) European Patent Application No. 0612850 (Nihon Millipore Kogyo KK).
(3) U.S. Pat. No. 5,258,285 (Foss Electric Holding AS)
(4) US Patent Application Publication No. 2008014607
(5) International Patent Application Publication No. WO92/02632 (Sierra Cytometry)
(6) Jones, D. L., M. A. Brailsford, and J.-L. Drocourt. 1999. Solid-phase, laser-scanning cytometry: a new two-hour method for the enumeration of microorganisms in pharmaceutical water. Pharmacop. Forum 25:7626-7645

### BACKGROUND OF THE INVENTION

Quantitative measurement of biological entities in samples, particularly liquid samples, is important in determining extent of contaminations, infections, state of a diseases etc. For example, in microbiology, the measure of bacterial or fungal number is provided by Colony Forming Unit (CFU/ml).

While microorganisms include forms of spores, inactive (non-culturable) and reproducible microorganisms, there is importance in having means for quantification of only reproducible microorganisms. For example, quantification of reproducible microorganisms is one of the main issues in the quality control processes of food and beverage industries, the manufacture of articles that come in contact with the consumer such as pharmaceuticals and personal care, environmental protection of aquatic systems such as rivers lakes and oceans, public safety (e.g., bacteriological monitoring of municipal water systems, wells, recreational waters such as pools spas and beaches etc.) various industrial processes, healthcare associated infections and the provision of medical care.

Quantification of dividing (culturable) microorganisms is typically a specialized laboratory procedure based upon a bacteria culture growing. Specific conditions for microbial growth on solid and liquid media need to be maintained over long incubation times at the end of which a CFU (CFU/ml) is determined. In the laboratory, the CFU is typically calculated by normalizing the total number of counted colonies according to the number of dilutions and the volume of the sample. This methodology requires laboratory equipment, qualified personnel and long time periods which may range from one day to one month. Notwithstanding the laborious and time consuming work involved therewith, CFU is the current standard accepted by regulatory bodies for microorganism counting.

The art provides various methods for the detection of microorganisms in tested samples which, as an alternative to cell counting, make use of color or fluorescent labeling.

For example, International patent application publication No. WO06/065350 (Kimberly Clark Worldwide Inc.) describes a method for semi-quantitatively or quantitatively detecting the presence of a microbe in a sample is provided. The method utilizes a test dye that undergoes a detectable color change in the presence of one or more microbes. For example, the test dye is a solvatochromic dye (e.g., Reichardt's dye) that responds to differences in polarity between microbe components (e.g., cell membrane, cytoplasm, etc.) and the environment outside the cell.

European patent No. EP0612850 describes a method of determining a viable microbial cell count in a sample solution, comprising filtering the sample solution through a filtration membrane having hydrophobic properties to entrap microbes within hydrophobic barriers; applying thereto a fine spray of ATP extracting reagent to extract a luminescent ingredient from the microbes; applying thereto another fine spray of liquid luminescence-inducing reagent for the luminescent ingredient extracted to allow the ingredient to emit luminescence; and measuring the level of the luminescence, using a competent means for measuring the luminescence level.

U.S. Pat. No. 5,258,285 describes a method for determining the number of bacteria in a cell population comprising bacteria and somatic cells.

US patent application publication No. US2008/014607 describes a bioluminescence-based method for detecting and counting living cells of a given species potentially present in a liquid sample, comprising measuring the total free intracellular adenyl nucleotides (AN) content, expressed in ATP form, of living cells of a given non-viral species.

International patent application publication No. WO92/202632 describes a process for the detection, identification, and/or enumeration of viable cells in bovine milk wherein viable cells are selectively labeled with a fluorescent dye (e.g. esterase-dependent dyes, nucleic acid binding dyes, and dyes that detect intracellular oxidative activity) and then identified and/or counted.

U.S. Pat. No. 7,312,073 describes a method for quantification of viable cells, where a sol-gel liquid precursor that incorporates a marker is transfixed as a thin layer coating on a slide. The slide is brought in contact for an incubation period with a filter containing microorganisms that were separated from a test sample. During the incubation period uptake of the marker/markers by the microorganisms occurs. The slide is then irradiated and the signal emitted from the marker contained microorganisms is detected to generate an image for the detection enumeration of the microorganism.

Further a two hour method for the enumeration of microorganisms in pharmaceutical water is described by Jones, et al (Pharmacop. Forum 1999, 25, 7626-7645).

### SUMMARY OF THE INVENTION

The present invention is based on the exploitation of cellular membrane dynamics according to which culturable (dividing) cells continuously undergo membrane trafficking. The inventors have now envisaged that in culturable microbial cells, cellular membrane is internalized, fused with internal membranous compartment and is then re-incorporated into the membrane at a rate that is much higher than in non-dividing cells.

The present invention is particularly based on the finding that staining microbial cells with a fluorescent dye that can associate with the membrane and internalize into the intracellular compartment of the microbial cell, results in the accumulation of the dye within the cell. This internalization process has a characteristic profile where after a first time period, T1, accumulation reaches a steady plateau and the accumulated amount remains steady at this plateau for a characteristic second time period, T2.

Yet further, the present invention is based on the finding that for a given microbial cell type or a group of microbial cells, the first time period T1 and the second time period T2 are characteristics to the cell(s) type. In other words, T1 and T2 will remain essentially constant for a given cell type (or group of cells) when measured under the same, predefined, conditions.

Thus, the present invention provides, according to a first of its aspects, a method for determining a quantitative value indicative of the number of culturable microbial cells in a tested sample, the method comprising:

(i) contacting the tested sample, susceptible of carrying culturable microbial cells with at least one signal emitting agent capable of associating with the microbial cell's membrane, said contacting is for a predetermined first time period (T1) being sufficient for internalization of the signal emitting agent into the microbial cell to a level at which the signal emitted from the sample essentially reaches a plateau;

(ii) removing from said tested sample non-internalized signal emitting agent;

(iii) during a second time period (T2), following said first time period (T1), and at which said signal is essentially maintained in said plateau, detecting from signal emitting objects within said sample signal emitting culturable cells, said detection being based on selection parameters predetermined for said culturable cells; and

(iv) determining, based on said selected signal emitting objects, a quantitative value indicative of the number of culturable cells in the tested sample.

The invention also provides a kit for determining a quantitative value equivalent to the number of culturable microbial cells in a tested sample, the kit comprising:

(i) at least one signal emitting agent capable of associating with a microbial cell's membrane,

(ii) instructions for contacting said at least one signal-emitting agent with the sample for a first time period (T1) being sufficient for internalization of the signal emitting agent into the microbial cell to a level at which the signal emitted from the sample essentially reaches a plateau;

(iii) instructions for removing from said sample non-associated signal emitting agent;

(iv) instructions for detecting and selecting from signal emitting objects within said sample signal emitting culturable cells, said detection being based on selection parameters predetermined for said culturable cells, the detection and selection being during a second time period (T2), following said T1, and at which said signal is essentially maintained in said plateau;

(v) instructions for use of said selected signal emitting objects for determining therefrom a quantitative value indicative of the number of culturable cells in the tested sample.

Yet further, the invention provides a system for determining a quantitative value indicative of the number of culturable microbial cells in a tested sample, the system comprising:

(i) a carrier for holding the sample and for permitting contacting a sample with one or more signal emitting agents;

(ii) a detector for detecting signal emitting objects within the sample, and outputing data corresponding thereto;

(iii) a memory unit comprising a database with predetermined selection parameters and a plurality of predetermined normalizing factors each selection parameter and each normalizing factor being specific for a microbial cell or for a group of microbial cells;

(iv) a processing unit for receiving the output data from the detector, one or more of said parameters and said normalizing factor from said memory unit and processing said output data with said parameters and said normalizing factor to determine said quantitative value indicative of the number of said culturable microbial cells in said sample.

Further provided by the invention is a program storage device readable by machine, tangibly embodying a program of instructions executable by the machine to perform a method for determining a quantitative value indicative of the number of culturable microbial cells in a tested sample, the method comprising:

(i) contacting said tested sample, susceptible of carrying culturable microbial cells with at least one signal emitting agent capable of associating with the microbial cell's membrane, said contacting is for a predetermined first time period (T1) being sufficient for internalization of the signal emitting agent into the microbial cell to a level at which the signal emitted from the sample essentially reaches a plateau;

(ii) removing from said tested sample non-internalized signal emitting agent;

(iii) during a second time period (T2), following said first time period (T1), and at which said signal is essentially maintained in said plateau, detecting and selecting from signal emitting objects within said sample signal emitting culturable cells, said detection being based on selection parameters predetermined for said culturable cells; and

(iv) determining, based on said selected signal emitting objects, a quantitative value indicative of the number of culturable cells in the tested sample.

In addition, provided is a computer program comprising computer program code means for performing all the steps of the invention, when said program is run on a computer, the computer program being embodied in a program storage device.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 is a graph showing fluorescence intensity as a function of time of samples comprising a mixture of municipal water derived bacterial cells (-▪-), isolated E. Coli (-♦-), and Ps. aeruginosa (-▴-), stained with the fluorescent dye FM1-43.

FIG. 2 is a graph plotting CFU/ml count of microbial cells in a sample vs. a quantitative measurement of the amount of microbial cells in a same sample, the quantitative measurement being obtained by the method of the invention.

FIGS. 3A-3B are graphs showing the correlation between CFU/ml count obtained by conventional method (Standard CFU/ml) and a CFU equivalent obtained in accordance with an embodiment of the invention (FIG. 3A) as well as the correlation between each value (CFU equivalent and conventional CFU/ml) per tested sample (FIG. 3B).

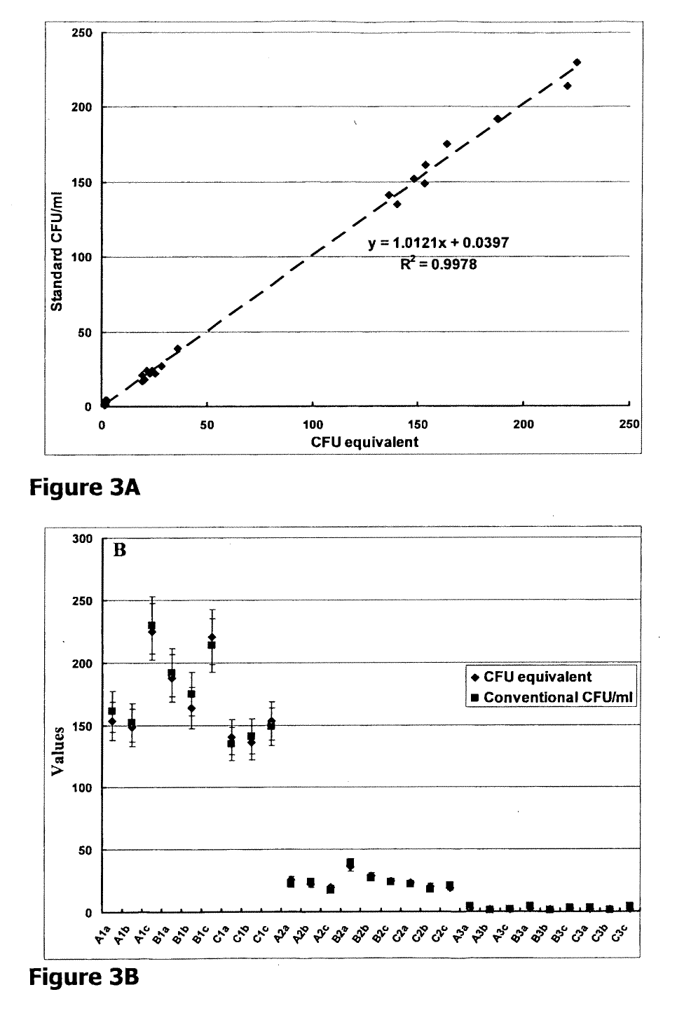

The results in Table 2 were also plotted in a graph, provided as FIG. 3A (♦ representing CFU equivalent, ▪ representing standard CFU), with the correlation between CFU equivalent and standard CFU presented in FIG. 3B.

### DETAILED DESCRIPTION OF THE INVENTION

Generally, the present invention provides methods, kits and systems for quantitatively measuring the amount of culturable microbial cells in a sample, typically a liquid sample, within several minutes after the sample is stained with a conventional signal emitting agent.

Conventional microbial cell counting techniques, such as the Heterotropic Plate Count (HPC) discussed below, typically require culturing the microbial cells for many hours in order to distinguish between dead cells and culturable microbial cells and counting only the cultured cells. Some of the culturable microbial cells are identified as Colony Forming Units (CFU/ml). Against this, the present invention allows within a few minutes the counting of only culturable microbial cells, to give a quantitative value that is equivalent to CFU. The invention, thus, allows for a CFU equivalent, while shortening the time and reducing the costs required as compared to conventional methods. While the CFU equivalent of the invention correlates with conventional colony forming unit measurements (such as HPC), some diversion may exists and any such diversion will not exceed a difference of more than 30%, preferably no more than 10% and more preferably, no more than 5%.

In the context of the invention the term “culturable microbial cell” or “culturable cell” is used to denote any cell (e.g. parent cell) of microscopic or ultramicroscopic size that when placed on a suitable and controlled media can divide into two (daughter) cells. Accordingly, the term “non-culturable microbial cell” denotes cells, even if viable, when placed in culturing conditions, do not and cannot divide into daughter cells.

The microbial cells may include, without being limited thereto, Bacteria, such as Coliforming bacteria (E. Coli), Enterobacteria (salmonella, listeria, shigella), Pseudomonas group (pseudomonas auriginosa, pseudomonas fluorescensa), Staphylococcus group (staphilococus aureus, streptococcus fecalis), Streptococcus group and Methanobacteria, etc.; Molds such as Aspergillus niger, penicillium group, etc.; Yeasts, such as Candida group, Sachramises group, etc.; Protozoa, such as Cryptosporidium group, Giardia group, Amoeba etc.; Algae such as green algae etc.; Acidophylic bacteria (TAB); legionella group; Vibrio species; and others.

The invention may have various applications, preferably but not exclusively, when there is a need for essentially immediate identification and quantification of microbial contaminations in samples, e.g. disease causing (pathogenic) agents. As an example, the method of the invention can be applicable for quantitative measuring the amount of microbial cells in drinking water. Some other applications may be related to industrial microbiology (e.g. cells present in food or beverage, personal care, industrial processes and healthcare associated infections), aquatic systems (potable water, process water, wastewater, natural water sources, recreational waters such as pools spas and beaches etc.), medical microbiology (plasma, saliva, urine, throat sample, gastrointestinal fluids), environmental microbiology (soil, air surfaces) etc.

As indicted hereinabove, the solution provided by the present invention for almost real time determination of the amount of culturable microbial cells in a sample is based on the exploitation of membrane trafficking, or movement of cell membrane to the intercellular compartment, that takes place in culturable cells to an extent that is, by a factor 1.5, 3, 6, 12 and even up to 20, greater than that occurring in viable, albeit, non-culturable cells. Non-culturable cells may include, for example, spores, anabiotic cells etc. As such, it was thus hypothesized by the inventors that if it is possible to stain a cell's membrane with a signal emitting agent that associates with the membrane and membrane trafficking occurs, then, the signal emitting agent will accumulate to a higher extent in the culturable cells as compared to non-culturable cells and thereby give rise to a stronger signal from these culturable cells.

The terms “essentially immediate” or “immediately” or “almost real time” denote a time window of less than 20 minutes, preferably, less than 15 minutes, more preferably, less than 10 minutes from initiating the method of the invention until at least one image of the tested sample is captured from which the CFU equivalent according to the invention can be deduced (typically and preferably by image processing), as will be further discussed below. In other words, the method of the invention is so instant it does not require the long term (hours) of culturing cells in order to determine the number of culturable cells in a tested sample.

Thus, in accordance with a first aspect, the invention provides a method for determining a quantitative value indicative of the cell count of culturable microbial cells in a tested sample, the method comprising:
(i) contacting the tested sample, susceptible of carrying culturable microbial cells, with at least one signal-emitting agent capable of associating with the microbial cell's membrane, said contacting is for a predetermined first time period (T1) being sufficient for internalization of the signal-emitting agent into the microbial cell to a level at which the signal emitted from the sample essentially reaches a plateau;
(ii) removing from said tested sample non-internalized signal emitting agent;
(iii) during a second time period (T2), following said first time period (T1), and at which said signal is essentially maintained in said plateau, detecting from signal emitting objects within said sample signal emitting culturable cells, the detection being based on selection parameters predetermined for the culturable cells; and
(iv) determining, based on said selected signal emitting objects a quantitative value indicative of the number of culturable microbial cells in the tested sample.

The method according to the invention comprises providing the sample susceptible of carrying culturable microbial cells with conditions that allow culturing of the cells. The conditions can be easily determined by those versed in the art based on available art and will typically depend on the type of microbial cells or group of microbial cells suspected of being present in the sample. The conditions may be, for example, similar or identical to those required for HPC.

The tested sample may be a liquid, semi liquid as well as a dry. When the sample is a liquid sample it may be dried before initiating the determination assay. The sample does not need to reach full dryness, but rather to remove at least a portion of the liquid therefrom, so as to concentrate the cells in the sample, to receive a cell concentrate.

Concentrating microbial cells in liquid samples may use any means available in biological laboratories. These include, without being limited thereto, filtering of the sample via suitable filters, centrifugation, and other drying techniques.

The sample (either as received or after removing therefrom at least a portion of the liquid) is stained with one or more signal emitting agents. The term “signal emitting agent” as used herein denotes any chemical entity that under appropriate conditions emits a detectable signal. The signal emitting agent may be a light emitting agent e.g. colorimetric agents, or a luminescence-emitting agent, the latter being, for example photoluminescence (including fluorescence or phosphorescence), chemoluminescence, radioluminescence, thermoluminescence; or any other signal emitting agent known in the art of cell labeling.

Examples of luminescent emitting moieties that can be used in accordance with the invention comprise, without being limited thereto, bioluminescents including luciferin based agents (e.g. 6-O-beta-galactopyranosyl luciferin), fluorescents including members of the Alexa Fluor family (Invitrogen), PromoFluor Dyes (PromoKine), HiLyte Fluors (AnaSpec), DyLight Fluors (Pierce, Thermo Fisher Scientific), and the ATTO Dye series (ATTO-TEC and Sigma-Aldrich). Those versed in the art should appreciate that a wide variety of fluorescent or other luminescent agents that can be used in accordance with the invention

In one embodiment, the agent comprises a luminescent, preferably, a fluorescent moiety conjugated to a lipophilic linker permitting the association, e.g. embedment of at least a portion of the agent with the culturable cells membrane and thereby internalization of the agent in the cell as a result of membrane trafficking.

The association of the signal emitting agent to the cell membrane of culturable cells is typically non-specific and is a result of the lipophilicity of the agent (e.g. due to the linkage of a luminescent moiety to lipophilic linker). Thus, a non-specific signal emitting agent is typically used for obtaining a total culturable bacterial count (TCBC).

The invention also allows for obtaining a cell count of a specific type of culturable microbial cell in a sample even having a mixture of microorganisms. This may be achieved using various specific signal emitting agents, either alone or in combination with a non-specific signal emitting agent.

The term “specific” or specificity” in the context of the term “specific signal emitting agent” is used to denote that the agent has affinity and/or selective binding to the membrane or to an intracellular component of a specific cell type (a cell, the detection of which in the sample is desired).

A specific signal emitting agent may comprise a targeting entity i.e. a ligand having binding specificity to an extracellular component of the cell. In one embodiment, the targeted signal emitting agent is such that it can internalize into the culturable cell, thereby allowing detection of only those that have internalized the signal emitting agent.

In another embodiment, the targeted (specific) signal emitting agent does not internalize into the cell to which it is targeted, and the detection of these cells is obtained by the combination of this specific signal emitting agent with a non=specific signal emitting agent. To this end, the non-specific signal emitting agent provides a total culturable bacterial count and the specific signal emitting agent provides a total count of a cell type (to which the signal specifically binds). The signals emitted from the images obtained with the two signal emitting agents are then superimposed so as to deduce from only those cells that emit both signals, i.e. that satisfy both criteria of being culturable and being capable of binding the specific agent.

Specificity of a signal emitting agent may also be achieved using enzymatic entities (e.g. where the enzyme activates intracellular reactions specific to a cell type). Such enzymes may include, without being limited thereto, those that act on ortho-nitrophenyl-beta-D-galactopyranoside to create a signal emitting degradation product. This may facilitate the identification of a specific cell population in a sample, having the specific enzyme that create the signal emitting degradation product

In yet a further example, the specificity (targeting) may be achieved by using a cell specific antibody (monoclonal as well as polyclonal). Examples of antibodies may include anti-salmonella LPS antibodies, anti CD18 antibodies, E. Coli LPS antibodies (e.g. E. coli J5 LPS antibody), Anti-Salmonella flagellum antigen Antibodies, Anti-E. Coli K99 attachment factor antibodies. As yet a further example, the antibody may be an immunoliposome (antibody linked to liposomes).

In yet another Example, the specificity may be achieved using bacteriophages (e.g. fluorescent-bacteriophage). Since the method of the invention provides a quantitative value within several minutes, it is possible to complete the assay before the fluorescent-bacteriophage damages the cells. As an example, E. Coli may be detected and quantified using bacteriophage Lamba Coliphage 1 (LG1)

An example of a combination of a non-specific and specific signal emitting agents may be, without being limited thereto, a luminescent moiety conjugated to cholera B toxin binding protein to detect Cholera Species, while E. Coli may be quantified using a luminescent moiety linked to an E. Coli specific membrane lipopolysaccharide, each being used in combination with a non-specific signal emitting agent such as FM1-43 used in the Examples.

It is noted that the agent can constantly emit a signal or the signal can be generated as a result of a stimuli, such as an enzymatic process taking place within the intracellular compartment, the enzymatic reaction manipulating the agent to emit the signal or the enzymatic degradation product emits a signal (as discussed above), a radiation stimuli, etc.

During the contacting stage, the signal emitting agent associates with the cells' membrane. In the context of the present invention the term “association” denotes any type of interaction of the agent with the cells' membrane; this including, without being limited thereto, electrostatic bonding, ionic bonding, covalent bonding, embedment of at least a portion of the marker in the cell's membrane, Antigen-Antibody bonding, Receptor-Ligand bonding and the like. In the context of the present invention the term “association” also encompasses any signal emitting agent that has already been internalized in the cell.

The contacting of the sample susceptible of carrying culturable microbial cells is for a time period sufficient for internalization of the signal emitting agent into the cell and sufficient for the intracellular accumulation of the signal emitting agent. It has been found by the inventors that, due to membrane trafficking occurring in culturable cells to a greater extent than in non-culturable cells, the culturable microbial cells have a higher degree of accumulated agent within the cell. The difference between non-culturable and culturable cell may be by a factor of 1.5, 3, 6, 12 and even up to 20 with respect to the amount of accumulated agent, under given conditions. It has been further determined by the inventors that the accumulation of the agent within the cell reaches a plateau.

The time required for the agent to reach a plateau is defined herein as “T1”. The time T1 is characteristic for a cell type and is pre-determined. Pre-determination of T1 can be achieved by staining a selected cell type with a signal emitting agent and detecting the change in signal intensity in time until there is essentially no change in the signal intensity. The time point from which there is no apparent change in the signal intensity is define as the time at which the plateau is reached. The term “plateau” as used herein denotes a level of intracellular accumulated signal emitting agent that remains the same for at least 50, 250 and even up to 900 seconds.

For the sake of illustration, reference is made to the examples provided herein which show that staining of sample containing isolated E. Coli with the non-specific signal emitting agent, FM1-43 (Molecular probes production) required a T1 of between about 90 to 110 seconds until the level of signal captured reached a steady state level; while for a sample containing isolated Ps. aeruginosa stained also with FM1-43 a T1 of between about 70 to 100 seconds was required for reaching a steady state level.

Since T1 is a priori determined for cells (or group of cells) susceptible of being in a tested sample, it is possible to determine a time point for washing out non-associated and preferably non-internalized signal emitting agent(s). Washing of the agent may be achieved by any technique known in the art. This may include, without being limited thereto, filtration (e.g. with a conventional microbial filter), centrifugation (e.g. above 4500 rpm) and any other technique that allows separation of the cells from the non-associated agent(s) dissolved or suspended in the liquid of the sample.

Cells having associated thereto a signal emitting agent are detected as signal emitting objects. As appreciated, however, the signal emitting agent may also associate with cell components or with other artifacts in the sample that is not of intact culturable cells and may provide a false signal. Similarly, the signal may be from aggregated signal emitting agent (i.e. that was not sufficiently dissolved in the sample). Thus, the method provides a tool for detecting and selecting only those signal emitting objects that originate from cells having associated thereto and internalized therein the agent. In the context of the present invention, the term “signal emitting object” denotes any optical spot from the tested sample that emits a signal. The signal emitting object does not necessary have the size of a cell, and in fact, may be larger, due to a halo around the cell formed from the signal emitted from the cell, especially when the imaged cell is out of focus (“circle of confusion”). This is caused by a cone of light rays from a lens not coming to a perfect focus when imaging a signal emitting cell The detection and selection is carried out after the signal reaches a plateau, at a second time period, T2. This time period, T2, represents the time window during which the signal emitted from the signal emitting object is retained at the plateau, essentially in a steady state.

During T2 several signal parameters are detected. These parameters include the intensity of the signal emitted from the signal emitting object, the size of signal emitting object within the tested sample, the morphology of the signal emitting object.

The selection criteria require for a microbial cell or group of microbial cells that the intensity of the signal emitted from the detected objects be at least below a predetermined upper threshold, and in some embodiments within a predetermined intensity range. Thus, signal intensity below a predetermined minimal level e.g. emitted from cell fragments (dead and damaged cells) or signal intensity above a predetermined maximal level (the upper threshold), e.g. emitted from aggregates of agent or from non-cellular bodies in the sample, is discarded.

The selection criteria also require that the signal emitting objects have a predetermined size range. This includes discarding objects below a minimal threshold (e.g. relating to cell fragments) or above a maximum threshold (e.g. from large bodies in the sample).

Further, the selection criteria may require that the signal emitting objects have a predetermined morphology. As appreciated, cell morphology is generally characteristic of a given bacterial species and such cells come in a wide variety of morphologies. These may include essentially rounded (e.g. Coci), essentially elongated (e.g. Bacilli), rod-like morphologies etc. The signal emitting object would typically have a shape corresponding to (resembling) that of the cell from which it is emitted. For example, an elongated signal emitting object would typically originate from an elongated microbial cell. The detection of the object's shape allows not only the discarding of non-microbial signal emitting objects (i.e. the artifacts) but may also facilitate in identifying the type of cell from which the signal is emitted.

In one embodiment, at least the intensity parameter and the size parameter need to be satisfied for detection and selection of the signal emitting objects.

It is noted that the selection parameters may vary between applications. For instance, for testing drinking water the ranges, thresholds and selected shapes may be different from those predetermined for testing water quality of, e.g. lakes.

The following no-limiting examples show that for a mixture of microorganisms comprising at least E. Coli and Ps. aeruginosa the size range was determined to be a diameter of 0.8-2 μm, and the intensity parameter was determined to be below 254 GL (and within the range 60-254 GL, not shown in the examples).

In one embodiment, at least two parameters, preferably the intensity parameter and the size parameter need to be fulfilled for identifying signal emitting objects. Once the signal emitting objects that fulfill the predetermined selection criteria are identified, these objects are selected and a quantitative value is deduced thereform. In one embodiment, their mean root square value of intensity is summarized; this summarized intensity being preferably normalized with a predetermined normalizing factor (e.g. by using a predetermined equation (see below Materials And Methods)), to obtain a quantitative value indicative of the number of culturable microbial cells in the sample (i.e. a CFU equivalent). The normalizing factor is specific to a cell type or for a group of cells, per the type of tested sample (i.e. per application) and is determined based on amounts of the cells in tested samples a priori measured under controlled conditions per application. For example, the normalizing factor is determined once for the drinking water of a geographical location under control conditions and by correlating values obtained from captured images as described with counts of cells obtained by conventional methods, such as the HPC(CFU/ml). This normalizing factor can then be used for future determination of the quality of the drinking water at that location (see Materials and Methods).

In accordance with one embodiment of the invention, a normaltization factor (equation) of 0.025x+0.3375 (x being the logarithmic scale of total fluorescence) was determined for a test sample comprising a mixture of E. Coli and Ps. aeruginosa.

The invention also provides a kit for determining a quantitative value equivalent to the number of culturable microbial cells in a tested sample, the kit comprising:
at least one signal-emitting agent capable of associating with a cell's membrane,
instructions for contacting said at least one signal-emitting agent with the sample for a first time period (T1) being sufficient for internalization of the signal-emitting agent into the cell to a level at which the signal emitted from the sample essentially reaches a plateau;
instructions for removing from said sample non-associated signal emitting agent;
instructions for detecting and selecting from signal emitting objects within said sample signal emitting culturable cells, said detection being based on selection parameters predetermined for said culturable microbial cells, the detection and selection being, during a second time period (T2), following T1, and at which the signal emitted from the sample is essentially maintained in said plateau;
instructions for use of said selected signal emitting objects for determining therefrom said quantitative value indicative of the number of culturable microbial cells in the tested sample.

The kit may also comprise instructions for removing from said sample at least a portion of the liquid prior to staining the sample with said signal emitting agent.

In accordance with one embodiment of the invention, the kit comprises a plurality of signal emitting agents. The plurality of agents being characteristic for an application, i.e. a kit dedicated for testing quality of municipal water for home use which will thus include agents specific for microbial cells that typically are found in municipal water.

In one embodiment, the instructions include guidelines for selecting cells or group of cells to be detected, and also includes a signal intensity upper threshold and/or signal intensity range predetermined for said cells or group of cells, a size range for the objects that will be detected, and predetermined signal emitting objects' morphologies and instructions for selecting those objects that satisfy the guidelines.

Further, in accordance with this particular aspect, the kit may comprise one or more pre-determined cell specific normalizing equations, each specific to a bacterial cell type, fungi or other microbial types type, and instructions for use of said pre-determined equation for deducing therefrom the CFU equivalent quantitative value.

The invention also provides a system for determining a quantitative value indicative of the number of culturable microbial cells in a tested sample, the system comprising
a carrier the for holding a sample and for permitting contacting of the sample with one or more signal emitting agents.
a detector for detecting signal emitting objects within the sample, and outputing data corresponding thereto;
a memory unit comprising a database with predetermined selection parameters and one or more predetermined cell specific normalizing factors, each selection parameter each normalizing factor being specific for a microbial cell or a group of microbial cells;
a processing unit for receiving the output data from the detector, and for receiving from said memory unit selection parameters and said normalizing factors specific for a microbial cell or a group of microbial cells and processing said output data with said parameters and said normalizing factor, to determine therefrom the quantitative value equivalent to the number of said culturable microbial cells in a sample.

The carrier may be any receptacle that can hold biological cells. In one embodiment, the carrier comprises a filter for filtering out at least a portion of liquid from said sample and holding the semi-dried or dried sample.

The detector in accordance with the invention typically comprises a luminescent imaging system, such as a camera being capable of capturing one or more images of luminescent signals emitted from the tested sample. Non-limiting examples of cameras that can be used in accordance with the invention include charge-coupled device (CCD), CMOS detector, photodiode (PD) detector, photomultiplier tube (PMT), gamma counters, scintillation counters or any other signal capturing device known in the art imaging of luminescent objects.

The image processing unit is configured to receive one or more images emitted from the tested sample and identify therefrom, based on the selection parameters of signal emitting objects, the number of culturable cells in the sample.

As used herein the term “processing unit” denotes any data processing and analyzing utility preprogrammed to collect measured signal parameters from the signal emitting objects in the sample and carry out data analysis consisting of selecting signal parameters according to predefined conditions and output a quantitative value based on the selected selection parameters. To this end, the processing unit carries a computer based program configured to carry out the analysis.

In one embodiment, the image processing unit is configured to select among the whole signal emitting objects, those that have a signal intensity within a predetermined range, a size within a predetermined range and a pre-determined morphology; and determined for the selected signal emitting objects the mean root square value intensity. The processing unit is further configured to output a quantitative value based on the mean root square value intensity emitted from the selected objects.

In yet a further embodiment, the image processing unit is configured to normalize the quantitative value with a normalizing factor retrieved from the database to obtain a normalized value that is equivalent to CFU/ml count.

The invention has been described in an illustrative manner, and it is to be understood that the terminology which has been used, is intended to be in the nature of words of description rather than of limitation. Obviously, many modifications and variations of the present invention are possible in light of the above teaching. It is therefore, to be understood that within the scope of the appended claims, the invention may be practiced otherwise than as specifically described hereinafter.

As used in the specification and claims, the forms “a”, “an” and “the” include singular as well as plural references unless the context clearly dictates otherwise. For example, the term “a signal parameter” includes one or more parameters.

Further, as used herein, the term “comprising” is intended to mean that the methods, kits and systems include the recited elements, but not excluding others. Similarly, “consisting essentially of” is used to define methods kits and systems include the recited elements but exclude other elements that may have an essential significance on the performance of the invention. “Consisting of” shall mean excluding more than trace elements of other elements. Embodiments defined by each of these transition terms are within the scope of this invention.

Further, all numerical values, e.g., concentration or dose or ranges thereof, are approximations which are varied (+) or (−) by up to 20%, at times by up to 10% of from the stated values. It is to be understood, even if not always explicitly stated that all numerical designations should be read as if preceded by the term “about”. It also is to be understood, although not always explicitly stated, that the reagents described herein are merely exemplary and that equivalents of such are known in the art.

### DESCRIPTION OF NON-LIMITING EXEMPLARY EMBODIMENTS

### General

In the following non-limiting example, samples comprising municipal water (tap water) mixed with predetermined concentrations of bacteria were used for determining the efficiency of the method of the present invention.

In each sample, the amount of viable reproducing microorganisms was quantified making use of the method of the invention as well as the standard CFU count (Standard Methods for Examination of Water & Wastewater (Lenore S. Clescerl (Editor), Arnold E. Greenberg (Editor), Andrew D. Eaton (Editor). 18-th Edition, 2002.). The results were compared and the efficacy of the method of the invention had a correlation of R²=0.997 to the CFU method.

### Materials and Methods

### Microbial Cells:

Three microbial cell stock preparations were provided:

Native forms of E. coli (1^{st} preparation) and Ps. aeruginosa (2^{nd} preparation) were isolated from tap water, using for E. Coli, Tryptone Bile X-Glucuronide (TBX) Medium (Promega production) and for Ps. aeruginosa Cetrimide agar (Promega production). A 3^{rd} preparation contained regular tap water and is referred to as the microbial mix preparation (as it typically would contain microbial mixute).

The cultured and isolated E. Coli and Ps. aeruginosa and the tap water preparatrions were each transferred into broth growing media and incubated by shaking using Lysogeny broth (LB) medium (Promega Cat. #7290A).

Incubation conditions for the three preparations:

E. coli −35° C. for 18 Hr.;

Ps. aeruginosa −30° C. for 40 Hr.;

Water microbial cell mix −30° C. for 72 Hr.

The cultured preparations were triple rinsed by centrifugation (6,000 RPM for 5 min) using Iso-normal PBS. At this stage microbial concentrations, as determined by microbial filtration CFU count, were determined to be about:

10¹⁰ CFU/ml for E. coli;

10⁹ CFU/ml for Ps. aeruginosa; and

10⁸ CFU/ml for water derived microbial mix.

Then, the microbial preparations (E. Coli and Ps. aeruginosa) were diluted with sterile PBS, while the tap water bacterial mix preparation was used as is or diluted with sterilized tap water (prepared by filtration of tap water via 0.22 μm microbial filters (Milipore production)). Heterotrophic Plate Count (HPC) was prepared using Microbial filtering (0.45 μm filters) procedure (Standard Methods for Examination of Water & Wastewater/By Lenore S. Clescerl (Editor), Arnold E. Greenberg (Editor), Andrew D. Eaton (Editor). 18-th Edition, 2002.) with testing volumes of suspensions in range from 0.01 ml to 1 Liter. Filters were incubated on PCA (Promega Cat. #7157A) medium with incubation conditions as follows:

E. coli −35° C. for 24 Hr.;

Ps. aeruginosa 30° C. for 48 Hr.;

Bacterial mix 30° C. for 72 Hr.

Results of CFU count were normalized to 1 Liter testing volume.

### Fluorescent Dye:

In the following experiments, FM1-43 styryl dye (Nishikawa S. Sasaki F. Internalization of styryl dye FM1-43 in the hair cells of lateral line organs in Xenopus larvae, Histochem Cytochem, 1996 44(7):733-41) was used at a concentration of 1 μg/ml in PBS.

### Staining Method:

Fluorescent staining of the microbial cell preparations was conducted at room temperature (about 25° C.) using FM1-43 styryl dye. Times of staining and working time window were determined using a monolayer preparation of the cells and it was determined that 2 minutes are required for the signal to reach the signal plateau (T1, see Determination of T1 and T2 below) and the plateau remained at steady state for a period of time of about 600 seconds (T1, see Determination of T1 and T2 below). At end of the staining the cultures were triple rinsed with iso-normal PBS. It is noted that the same staining method was used for filters surface adhered cultures or for suspended cultures (i.e. this is not specific for monolayer preparations).

### Fluorescence Imaging:

Fluorescent images of the signal emitting samples were obtained using Axiovert 200 Microscope (Karl Zeiss), 1.3NA Plan Neofluor X10 objective (BP 450-490 excitation filter (Excitation: 450-490 nm; Beam splitter: FT 510 nm; Emission: 515-565 nm; Karl Zeiss).

### Image Capturing:

The images were captured making use of a standard Sensicam qe (Cooke Corp.) 512×512 CCD.

### Image Processing:

Image processing and data collection were performed using ImagePro+software (2002).

### Determination of T1 and T2:

For determining of T1 three samples were prepared from the stock preparations, i.e. a tap water sample comprising a bacterial mix naturally existing in the tap water (after being cultured on a culturing medium and washed with PBS as described above); a second sample of isolated E. coli (after being cultured, and washed with PBS, as described above) and a third sample of isolated Ps. aeruginosa (after being cultured, and washed with PBS, as described above).

A sample from each stock preparation was analyzed according to HPC methodology, to obtain the corresponding conventional CFU/ml count.

Each sample was then diluted with PBS to obtain samples of about 1000 CFU/ml concentration. Each sample was then stained with FM1-43 and imaged when in monolayer (as described above) every 10 seconds until the signal emitted from the monolayer reached a plateau. The time at which the signal reached a plateau was determined as T1.

### Determination of T2

In a further set of three such samples diluted from the three preparations, about 110 seconds following staining of the samples, the samples were washed with PBS, and imaged in monolayer in a time sequence of 10 seconds, until the signal started to fade. The time point at which the signal started to fate was determined as T2.

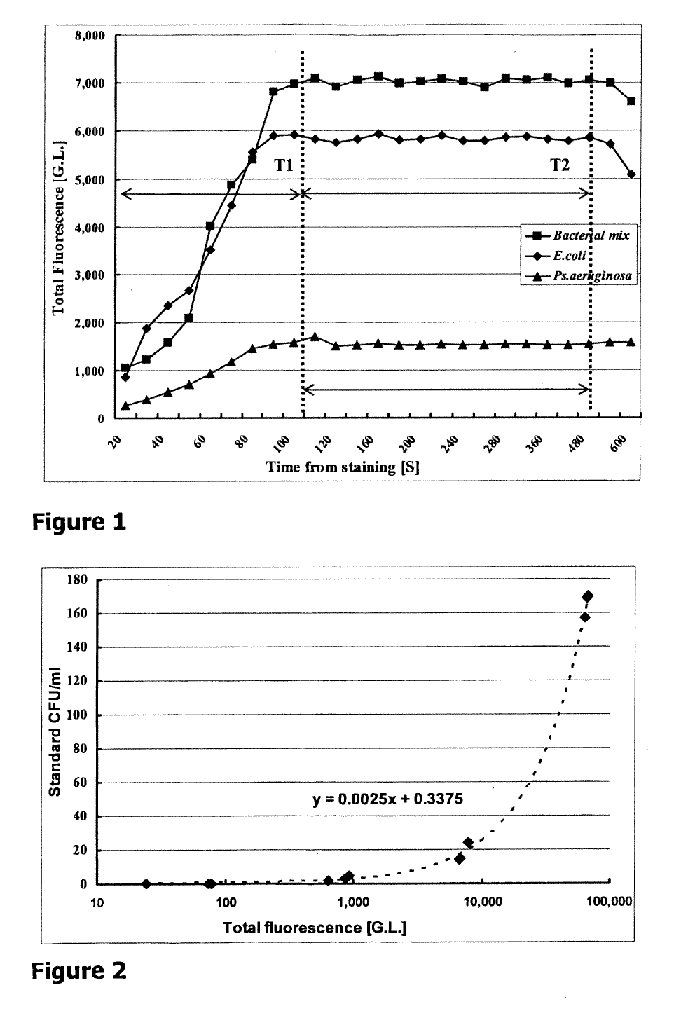

The above procedure was repeated three times. The intensity of the signal captured during the measurement session was plotted and the results are shown in FIG. 1.

Determination of CFU equivalence:

In order to identify a CFU equivalent (i.e. to calibrate the measurement of the invention with the standard CFU/ml count) a total of three samples of tap water from three different geographical locations (A, B and C) were collected and used. Preparations from each location were prepared as described above with respect to the determination of T1, to obtain isolated E. Coli and Ps. aeruginosa samples and a bacterial mix sample. For each location the concentration of each bacterial cell or the total bacterial count for each preparation was a priori determined based on HPC methodology (i.e. to obtain TBC before dilution) and then samples from the bacterial mix preparations (i.e. from each location, in total 3) were diluted with sterilized water to obtain four approximate concentrations, 1000 CFU/ml, 100 CFU/ml, 10 CFU/ml and 1 CFU/ml, i.e. in total 12 samples.

Each of the 12 samples was analyzed according to HPC methodology and also according to the method of the invention. In the latter case, each sample was stained with FM1-43 as described above, washed after 120 seconds with sterilized water (i.e. at the beginning of the Plateau) and immediately imaged (at about 150-180 seconds). The image was processed according to signal selection parameters. Specifically, once image were captured, objects satisfying selection parameters were numbered, and their mean square root intensity determined. The results of the selection are provided in Tables 1A-1B below.

The E. Coli and Ps. aeruginosa isolated from the samples prepared above were treated exactly as the bacterial mix sample. It was observed that the dynamics of changes were similar to those of the bacterial mix, and that there were no significant changes in the signals obtained from the bacterial mix sample of similar concentrations (data not shown). Validation of the method of the invention

A total of 48 microbial test samples were prepared from the three different locations as described above (4 samples from each location, each sample in triplicate). Specifically, preparations from each location (with unknown cell concentration) either diluted with sterilized water ×10, ×100, or non-diluted. Also, from each location, one sample of stock preparation was sterilized by filtration with a 0.22 μm mesh filter (Nitrocellulose, Milipore production).

Each test sample was then stained with FM1-43, washed with sterilized water and analyzed using the conventional HPC method or the method of the invention as described above. The results are shown in Table 2 below.

### Fluorescence Measurement

All fluorescence measurements and corresponding CFU counts were conducted at room temperature (˜25° C.).

### Imaging and Signal Processing

For the purpose of image analysis, a microbial signal emitting object (i.e. an object to be counted for) was determined to be of sizes 0.8-1.5 μm when visualized by light microscope corresponding to 12-28 pixels (in the particular set up used in these non-limiting examples). These were the selection criteria.

In the non-limiting examples provided herein, unless otherwise stated, the samples were imaged 150 to 180 seconds following staining (i.e. shortly after the 2 minutes following staining). Further, exposure time was calibrated for maximal fluorescent intensity in “microbial objects” to be 254 gray levels (or between 60-254 GL). Exposure time was adjusted to 0.8 seconds.

Averages calculation and Standard deviation (in percents) were used for determined the quantitative values.

### Results

The invention is based on the understanding that reproducing (culturable) microbial cells can internalize lipid fractions of extracellular cell wall and concentrate the same inside the cell for a period of time (faster as compared to non-culturable microbial cells). The membrane trafficking rate and its intracellular concentration level is a function of cell activity, very high in reproducible microorganisms. Thus, it has been envisaged by the inventors that this phenomena can be utilized in order to distinguish and quantify the amount of viable, reproducible cells in a sample.

### Determination of T1 and T2:

FIG. 1 shows that accumulation of FM1-43 until the signal reached a plateau was at about 120 seconds following staining. This time point was thus marked as T1. Stable intracellular dye accumulation was maintained steady in a time window of from 115 to 550 seconds from the beginning of the plateau. In other words, the fluorescent dye accumulation at the intracellular compartment of the mixture of cells became stable after 115 seconds and remained as such for at least 550 seconds. It has thus been determined that for quantitative determination E. Coli and/or Sp. Aeruginosa in a liquid sample, a time window of from about 115 to about 550 seconds is an effective measurement window T1.

### Determination of CFU Equivalence:

The images for the 12 microbial samples used for determination of the CFU equivalence, as described above, were processed to select there from objects satisfying the predetermined selection criteria (i.e. sizes 0.8-1.5 μm when visualized by light microscope corresponding to 12-28 pixels, maximal intensity of 254 gray levels).

The determined mean square root values of intensity for the selected objects are included in Tables 1A-1B together with the CFU/ml counts obtained by the conventional HPC methodology. The approximate concentration was determined based on the CFU/ml count obtained for the stock preparations. The different samples are identified by location (A, B, or C) and sample number per location (X1, X2 or X3).

TABLE 1A
Analysis of bacterial mix sample

| | | | No. of | Conventional |
|---|---|---|---|---|
| Approx. | Sample | | selected | CFU/ml |
| concentration | identifier | Intensity (GL) | objects | count |
| 1000 | A1 | 64,195 | 599 | 157 |
| 1000 | B1 | 67,375 | 624 | 170 |
| 1000 | C1 | 65,772 | 628 | 169 |
| 100 | A2 | 6,797 | 62 | 15 |
| 100 | B2 | 7,818 | 71 | 24 |
| 100 | C2 | 6,608 | 60 | 14 |
| 10 | A3 | 878 | 8 | 3 |
| 10 | B3 | 946 | 9 | 5 |
| 10 | C3 | 639 | 6 | 2 |
| 1 | A4 | 73 | 0 | 0 |
| 1 | B4 | 78 | 0 | 0 |
| 1 | C4 | 24 | 0 | 0 |

TABLE 1B
Analysis of E. Coli or Ps. Aeruginosa

| | | | No. of | |
|---|---|---|---|---|
| Microbial | Approx. | | selected | Conventional |
| Cell type | concentration | Intensity (GL) | objects | CFU/ml count |
| E. coli | 1000 | 48,572 | 641 | 336 |
| E. coli | 100 | 5,827 | 77 | 45 |
| E. coli | 10 | 543 | 8 | 3 |
| E. coli | 1 | 42 | 1 | 0 |
| Ps. aerugin. | 1000 | 18,366 | 167 | 88 |
| Ps. aerugin. | 100 | 1,660 | 15 | 9 |
| Ps. aerugin. | 10 | 118 | 2 | 10 |
| Ps. aerugin. | 1 | 7 | 0 | 5 |

The results provided in Table 1A are also presented in FIG. 2 which shows a correlation between CFU/ml values obtained by standard HPC method and the quantitative values obtained by the method of the invention. FIG. 2 specifically shows that the correlation between the two measuring methods can be presented by the following equation:

y=0.025x+0.3375,

This equation was thus determined to be the normalization factor for quantitative determination of microbial mix in tap water samples from municipal water systems.

### Validation of CFU Equivalent Method

To validate the accuracy of the above equation with respect to unknown tap water samples, a further assay was conducted with samples from municipal water from different locations as described above, however, without a priori determining bacterial concentration. From each location three samples were taken. All samples were diluted as described in the Materials and Methods (preparation for validation step) and tested in parallel for determining CFU equivalent count according to the method of the invention making use of the above identified normalizing equation, and standard CFU count. It is noted that the samples are identified by their location, dilution and sample number from triplicate set, i.e. for each location A, B or C, and for each dilution e.g. A1, A2, A3, A4, triplicates were used, A1a, A1b, A1c. Thus, for example, sample Ala denotes one of the three samples from location 1, with no dilution.

The results are presented in Table 2 providing a comparison between CFU equivalent count (the method of the invention) and standard CFU count of various the liquid samples.

TABLE 2
CFU equivalent and standard CFU counts

| | | Total | | Standard |
|---|---|---|---|---|
| | Sample | intensity | CFU | CFU/ml |
| Dilution | identifier | (GL) | equivalent* | count |
| 1:1 | A1a | 61,286 | 153.5525 | 161 |
| 1:1 | A1b | 59,178 | 148.2825 | 152 |
| 1:1 | A1c | 89,787 | 224.805 | 230 |
| 1:1 | B1a | 74,897 | 187.58 | 192 |
| 1:1 | B1b | 65,423 | 163.895 | 175 |
| 1:1 | B1c | 88,002 | 220.3425 | 214 |
| 1:1 | C1a | 55,987 | 140.305 | 135 |
| 1:1 | C1b | 54,254 | 135.9725 | 141 |
| 1:1 | C1c | 61,120 | 153.1375 | 149 |
| 1:10 | A2a | 10,021 | 25.39 | 22 |
| 1:10 | A2b | 8,547 | 21.705 | 24 |
| 1:10 | A2c | 7,589 | 19.31 | 17 |
| 1:10 | B2a | 14,250 | 35.9625 | 39 |
| 1:10 | B2b | 11,240 | 28.4375 | 27 |
| 1:10 | B2c | 9,520 | 24.1375 | 24 |
| 1:10 | C2a | 9,054 | 22.9725 | 22 |
| 1:10 | C2b | 8,041 | 20.44 | 18 |
| 1:10 | C2c | 7,560 | 19.2375 | 21 |
| 1:100 | A3a | 758 | 2.2325 | 4 |
| 1:100 | A3b | 658 | 1.9825 | 1 |
| 1:100 | A3c | 589 | 1.81 | 2 |
| 1:100 | B3a | 890 | 2.5625 | 4 |
| 1:100 | B3b | 509 | 1.61 | 1 |
| 1:100 | B3c | 787 | 2.305 | 3 |
| 1:100 | C3a | 587 | 1.805 | 3 |
| 1:100 | C3b | 541 | 1.69 | 1 |
| 1:100 | C3c | 657 | 1.98 | 4 |

*determined using the above identified equation: y = 0.025x + 0.3375

As shown, the difference between the quantitative value obtained by the method of the invention, i.e. the CFU equivalent, and the value obtained by the conventional HPC method was less than 10% for all tested samples. The correlation was found to be R²=0.9978, which confirms that the CFU equivalent count according to the method of the invention can be safely used for almost real time CFU count.

The results in Table 2 were also plotted in a graph, provided as FIG. 3A (♦ representing CFU equivalent, ▪ representing standard CFU), with the correlation between CFU equivalent and standard CFU presented in FIG. 3B.

### Comparisons of Determination of Standard CFU Count and CFU Equivalent for Reproducible Microorganisms Quantification

The CFU equivalent according to the invention provides means for a fast, and almost real time quantification of microorganisms in a liquid sample. Not only that the CFU equivalent is less time consuming as compared to standard CFU count methods, the time to receive the results in almost real time (between 5 to 10 minute, as compared to days when using the standard CFU count.

Furthermore, obtaining CFU equivalent count would be less costly, can be an automated process, it does not require large working spaces and can be determined close to tested area. The results of comparison are shown in FIG. 3A and in FIG. 3B. Specifically, FIG. 3A show that there is almost a liner correlation between the two measurement techniques, i.e. R²=0.9978, and FIG. 3B shows that for each sample, the values obtained by each method (HPC or the method of the invention) almost superimpose. Thus, it was concluded that the method of the invention can safely provide a CFU equivalent value.

1. A method for determining a quantitative value indicative of the number of culturable microbial cells in a tested sample, the method comprising:
(i) contacting the tested sample, susceptible of carrying culturable microbial cells with at least one signal-emitting agent capable of associating with the microbial cell's membrane, said contacting is for a predetermined first time period (77) being sufficient for internalization of the signal-emitting agent into the microbial cell to a level at which the signal emitted from the sample essentially reaches a plateau;
(ii) removing from said tested sample non-internalized signal emitting agent;
(iii) during a second time period (T2), following said first time period (T1), and at which said signal is essentially maintained in said plateau, detecting from signal emitting objects within said sample signal emitting culturable cells, said detection being based on selection parameters predetermined for said culturable cells; and
(iv) determining, based on said selected signal emitting objects, a quantitative value indicative of the number of culturable cells in the tested sample.

2. The method of claim 1, comprising removing at least a portion of liquid from said tested sample prior to staining the sample with said signal emitting agent.

3. The method of claim 2, wherein the removal of liquid is by one or more of filtration or centrifugation.

4. The method of claim 1, wherein the sample comprises one or more types of microbial cells.

5. The method of claim 1, wherein the signal-emitting agent is a luminescent agent or a light emitting agent.

6. The method of claim 1, comprising contacting said sample with at least one additional signal emitting agent that has specificity to a selected microbial cell suspected of being in said sample.

7. The method of claim 6, comprising detecting signal emitted from each signal emitting agent from said sample and determining therefrom a quantitative value indicative of the number of culturable specific microbial cells in the sample.

8. The method of claim 6, for determining a plurality of quantitative values, each value being indicative of the number of a specific microbial cell or of microbial cells of specific group of in the sample.

9. The method of claim 1, wherein said selection parameters comprise two or more parameters selected from intensity of signal emitted from a signal emitting object, size of the signal emitting object, morphology of the signal emitting object.

10. The method of claim 9, wherein only signal emitting objects having at least a signal intensity below a predetermined upper threshold and a size within an object size range are detected.

11. The method of claim 9, wherein only signal emitting objects having at least a signal intensity within a predetermined intensity range, and a size within an object size range are detected.

12. The method of claim 9, comprising calculating for said selected objects said quantitative value.

13. The method of claim 12, wherein said quantitative value comprises a mean square root intensity of said selected objects.

14. The method of claim 12, wherein said quantitative value is normalized to correspond with colony forming units (CFU) count, the normalized quantitative value being a CFU equivalent.

15. The method of claim 14, wherein the normalization comprises factoring said quantitative value with a pre-determined cell specific normalizing factor to obtain said CFU equivalent.

16. A kit for determining a quantitative value equivalent to the number of culturable microbial cells in a tested sample, the kit comprising:
(i) at least one signal-emitting agent capable of associating with a microbial cell's membrane,
(ii) instructions for contacting said at least one signal-emitting agent with the sample for a first time period (T1) being sufficient for internalization of the signal-emitting agent into the microbial cell to a level at which the signal emitted from the sample essentially reaches a plateau;
(iii) instructions for removing from said sample non-associated signal emitting agent;
(iv) instructions for detecting and selecting from signal emitting objects within said sample signal emitting culturable cells, said detection being based on selection parameters predetermined for said culturable cells, the detection and selection being during a second time period (T2), following said T1, and at which said signal is essentially maintained in said plateau;
(v) instructions for use of said selected signal emitting objects for determining therefrom a quantitative value indicative of the number of culturable cells in the tested sample.

17. The kit of claim 16, comprising instructions for removing from said sample at least a portion of the liquid prior to staining the sample with said signal emitting agent.

18. The kit of claim 16, comprising more than one signal emitting agent.

19. The kit of claim 16, wherein the signal-emitting agent is a luminescent agent or a light emitting agent.

20. The kit of claim 19, wherein said signal emitting agent is a fluorescent dye.

21. The kit of claim 16, comprising selection parameters and instructions for use of at least two of said selection parameters for selecting from signal emitting objects within said sample signal emitting culturable microbial cells.

22. The kit of claim 21, wherein said selection parameters comprise a predetermined signal intensity upper threshold, a signal intensity range, a size within a predetermined signal-emitting object size range and a signal emitting morphology.

23. The kit of claim 22, comprising instructions for use of at least two of said selection parameters for selecting only signal emitting objects with a signal intensity below a predetermined upper threshold or within an object size range, and having a size within a predetermined size range.

24. The kit of claim 15, comprising instructions for calculating from said selected objects said quantitative value.

25. The kit of claim 24, wherein said instructions comprise calculating a mean square root intensity of said selected objects to obtain therefrom said quantitative value.

26. The kit of claim 16, comprising instructions for normalizing said quantitative value with a normalization factor to obtain a colony forming unit (CFU) count equivalent.

27. The kit of claim 26, comprising one or more pre-determined normalizing factors and instructions for use of said pre-determined normalizing factors, for deducing from the measured quantitative value said CFU equivalent.

28. A system for determining a quantitative value indicative of the number of culturable microbial cells in a tested sample, the system comprising:
(i) a carrier for holding the sample and for permitting contacting a sample with one or more signal emitting agents;
(ii) a detector for detecting signal emitting objects within the sample, and outputing data corresponding thereto;
(iii) a memory unit comprising a database with predetermined selection parameters and a plurality of predetermined normalizing factors each selection parameter and each normalizing factor being specific for a microbial cell or for a group of microbial cells;
(iv) a processing unit for receiving the output data from the detector, one or more of said parameters and said normalizing factor from said memory unit and processing said output data with said parameters and said normalizing factor to determine said quantitative value indicative of the number of said culturable microbial cells in said sample.

29. The system of claim 28, wherein said carrier comprises a filter for filtering out at least a portion of liquid from said sample.

30. The system of claim 28, wherein said detector comprises a camera for capturing an image of the signal emitting objects within said tested sample.

31. The system of claim 30, wherein said processing unit is configured to determine, from the data received from said detector, for each signal emitting object detected intensity of signal, size of signal emitting object, morphology of signal emitting object.

32. The system of claim 28, wherein said processing unit is configured to select signal emitting objects having a signal intensity below a predetermined intensity threshold and a size within a predetermined size range.

33. The system of claim 28, wherein said processing unit is configured to select signal emitting objects having a signal intensity within a predetermined intensity range and a size within a predetermined range.

34. The system of claim 32, wherein said processing unit is configured to output a quantitative value, based on mean square root of the intensity emitted from the selected signal emitting objects.

35. The system of claim 28, wherein said processing unit is configured to normalize the quantitative value with a normalizing factor retrieved from said database.

36. The system of claim 28, comprising a cassette of one or more signal emitting agents, the system being pre-programmed to permit said cassette to release said one or more signal emitting agents into said carrier and to permit contact of said one or more signal emitting agents with a tested sample in said carrier.

37. The system of claim 28, comprising an array of cassettes, each carrying a signal emitting agent, the system being pre-programmed to permit release into the carrier of one or more signal emitting agents from said array of cassettes.

38. A program storage device readable by machine, tangibly embodying a program of instructions executable by the machine to perform a method for determining a quantitative value indicative of the number of culturable microbial cells in a tested sample, the method comprising:
(i) contacting said tested sample, susceptible of carrying cultivable microbial cells with at least one signal-emitting agent capable of associating with the microbial cell's membrane, said contacting is for a predetermined first time period (T1) being sufficient for internalization of the signal-emitting agent into the microbial cell to a level at which the signal emitted from the sample essentially reaches a plateau;
(ii) removing from said tested sample non-internalized signal emitting agent;
(iii) during a second time period (T2), following said first time period (T1), and at which said signal is essentially maintained in said plateau, detecting and selecting from signal emitting objects within said sample signal emitting culturable cells, said detection being based on selection parameters predetermined for said culturable cells; and
(iv) determining, based on said selected signal emitting objects, a quantitative value indicative of the number of culturable cells in the tested sample.

39. The program storage device of claim 38, executable by said machine to perform a method.

40. A computer program comprising computer program code means for performing all the steps of claim 1, when said program is run on a computer, the computer program being embodied in a program storage device.
